(19) United States
(12) Patent Application Publication
(10) Pub. No.: US 2018/0326169 A1
(43) Pub. Date: Nov. 15, 2018

(54) ENDOTRACHEAL CLEANING SUCTION BRUSH

(71) Applicant: Cedars-Sinai Medical Center, Los Angeles, CA (US)

(72) Inventor: Axel ROSENGART, Los Angeles, CA (US)

(73) Assignee: Cedars-Sinai Medical Center, Los Angeles, CA (US)

(21) Appl. No.: 15/772,703

(22) PCT Filed: Nov. 4, 2016
(86) PCT No.: PCT/US2016/060661

§ 371 Date: May 1, 2018

Related U.S. Application Data
(60) Provisional application No. 62/251,408, filed on Nov. 5, 2015.

(51) Int. Cl. A61M 16/04; A61B 90/70; A61B 1/12

(52) U.S. Cl. CPC A61M 16/0463; A61M 16/0402; A61M 16/0465; A61B 2090/701; A61B 1/122; A61M 2209/10; A61B 90/70

(57) ABSTRACT

The disclosed systems and methods provide a simple, safe, effective and widely applicable method to clean the inside of an endotracheal tube. For instance, disclosed is an endotracheal suction catheter that includes a brush-suction segment that allows the catheter to mechanically brush off secretions on the inner surface of an endotracheal tube (“ETT/TT”) while simultaneously suctioning the debris.

### FIELD OF THE DISCLOSURE

The present disclosure relates to devices and methods for suctioning of endotracheal tubes and upper airways. More particularly, the present disclosure relates to a device, a method, and a system for clearing the inside of the endotracheal tube using a combination of suction and a mechanical feature.

### BACKGROUND OF THE DISCLOSURE

The following description includes information that may be useful in understanding the present disclosure, it is not an admission that any of the information provided herein is prior art or relevant to the present disclosure, or that any publication specifically or implicitly referenced is prior art.

Patients in need of breathing support are generally treated with placement of a breathing tube (endotracheal tube (ETT)) through the mouth or nose into the windpipe, a process called endotracheal intubation. This method is generally utilized in the acute stages of respiratory failure; for example, in patients with acute pneumonia or brain injury. In contrast, patients in demand of chronic ventilatory support (i.e., ALS or post head trauma) receive a surgical opening in front of the neck (tracheotomy or tracheostomy) providing access to the windpipe via a tracheal tube (TT).

However, in either breathing support scenario, within hours after tube insertion; lung secretions (mucous) or blood, vapor from humidified breathing gas (moisture), and bacteria (seeding and generation of slimy microfilms) begin to increasingly lead to build-up along the inner surface of the breathing tube. Over days, such build-up becomes not only the perfect bacterial breeding ground but it also dries out and hardens to a solid film.

This build-up in the inner lumen for the endotracheal tube has, among others, two important detriments: (1) it further accelerates bacterial overgrowth and continuous bacterial spillage into lower lung segments leading to ventilator associated pneumonia (VAP); and (2) the inner tube diameter narrows over several days, leading to increased air flow resistance and hence, increased work of breathing through the tube which has been shown to delay respiratory recovery and independence (weaning delay). Evidence identifies that both bacterial seeding and luminal narrowing correlate to increased length of hospital stay and health care costs per patient.

Currently available technologies to clean the endotracheal tube is limited to “wipers” to clean the inner tube lining that include a pipe-like device with a grip, handle and pulling mechanism on the operator's end which, if pulled, allows a squeezable silicone plate at the opposite end to flatten and extend in diameter (Endoclear). To employ this device, the patient is temporarily disconnected from the breathing apparatus and the Endoclear device inserted into the ETT/TT to a predefined depth (which matches the total length of the ETT/TT) in order to position the expandable silicone plate at the inner (inside or tracheal) end of the ETT/TT.

Next, the device handle is pulled, expanding the silicone plate and leading to effacement and occlusion of the silicone against the inner ETT/TT wall. Then the silicone is slowly pulled out through the entire length of the ETT/TT performing the desired cleaning action by wiping the inner lumen of the entire ETT/TT.

Other alternatives using in-line or external catheters to suction the internal catheters. However, the suction catheters do not remove build-up inside the ETT/TT tube. Accordingly, the wiper device must be used separately and in conjunction with a suction catheter to remove all of the build-up, all the while. This separate suction and wiping puts additional burdens and interference on the patient's breathing circuit. Furthermore, ETT/TT wiping employing the Endoclear device is inherently associated with several risks including the following:

### (i) Risk of Drop in Blood Oxygen

For the time period required for ETT/TT cleaning the patient must be separated from the breathing circuit, especially from oxygen supply. Even though this disconnection time period may be short (˜1 minute; likely with a significant variation in disconnection time as this cleaning methods is operator-dependent) a significant number of patients with severe lung injury will not tolerate being disconnected at all from oxygen supply, therefore markedly increasing the risk of oxygen drop and hence, limiting Endoclear use in this patient population.

### (ii) Risk of Dislodgement of ETT

The expanded silicone cleaning ring of the Endoclear technology effaces highly against the inner ETT/TT and is pulled back against friction force in order to clear the ETT/TT—this process can lead to ETT/TT dislodgement and dangerous loss of airway in case the operator does not tightly anchor the ETT/TT against the Endoclear pulling movement.

(iii) Risk of Contamination

Because the Endoclear device is separate piece of equipment not integrated into the patent's breathing circuit and it can accidentally become contaminated prior to introduction of the device into the patient's airway. Introduction of dangerous pathogens can lead to marked pulmonary complications and death.

An unfilled need exists for a device, a method, and a system to clear the inside of the endotracheal tube in a safe and effective manner.

### SUMMARY OF THE DISCLOSURE

### Overview

The implementation of the presently disclosed devices and methods will result in measurable outcome improvements such as reducing acquired hospital complications (aspiration events, ventilator-associated pneumonia), patient comfort and autonomy, reduction in caretaker time, reduction in daily suction sets, among others.

Accordingly, a cleaning catheter is disclosed that includes both suction and a mechanical feature for removing debris build up on the inside of endotracheal and tracheal tubes. The combination allows a caregiver to, effectively, intermittently and on-demand, remove secretions from the endotracheal/tracheal tube and upper airways while simultaneously cleaning the inner surface of the ETT/TT to keep it free from bacterial overgrowth and secretion build-up.

In some embodiments, the combination cleaning catheter includes a suction-brush segment to the suction catheter which allows the ETT/TT brush cleaning with immediate aspiration of brushed off endoluminal debris each time the patient secretions are removed by conventional in-line suctioning. Conventional in-line suctioning is standard of pulmonary care and performed in almost all patients with ETT/TT at various frequencies depending on the underlying lung illness (i.e., from every 15 to 30 mins [or more often] to a few times per day). In some embodiments, the total segment length occupied by the brush/suction unit may be about 1 inch, 2 inches, 1.5 inches or other suitable lengths and fit within the length of the in-line suction tube.

The suction-brush segment may be incorporated into the actual suction tube and located at a point where when the suction tube is fully inserted into the ETT/TT, the suction-brush segment will be flush or at the distal tip of the ETT/TT. This position allows the brush segment to move through and clean the entire ETT/TT length while suctioning.

The suction-brush segment may include a brush portion with brush hairs that emanate from the tube. The brush portion may include two conically shaped, flexible brushes opposing each other at their larger bases. The conical shape permits smooth ETT/TT entry, exit and reentry.

The hairs of the brush may be aligned in four spiral arrays. Each array may be oriented to form a half circular turn pattern from the brush tip to its base. The spiral brush hair formation will provide lateral (circumferential) cleaning traction to the brush action in addition to the back-and-forth cleaning motions, which occurs during the insertion and retrieval motion during suctioning.

The length of the brush hairs would be slightly longer than the remaining space between the suction tube and the inner ETT/TT diameter. For example, the brush hairs may be about ⅛^{th}, 3/16^{th}, or 1/16^{th} of an inch in some embodiments. This brush length allows brushing with some resistance against the inner ET/TT surface in order to effectively remove bacterial films and secretions.

In some embodiments, the brush hair arrays may have small suction channels (grooves within the suction tube) spaced in-between and adjacent to the spiral brush arrays. These perforations allow aspiration of debris/secretions into the suction tube. To optimize debris/section aspiration and to minimize overall suction pressure loss within the in-line suction device, the channels may be grooved in a V-form (with the larger channel width at the outer and smaller width at the inner wall of the suction tube). This allows the creation of a pressure gradient with lower suction pressure towards the brush surface while higher suction pressure is generated at the inner suction tube lumen.

When a caregiver inserts the cleaning tube in the ETT/TT, brush action and cleaning are automatically performed whenever the suction system is utilized. Generally, care goals demand one tracheal suctioning every hour in ventilated patients; however, many patients require much more frequent suctioning. Therefore, utilizing the proposed brush segment, a patient's ETT/TT tube is cleaned (that is brushed with simultaneous aspiration of debris/secretions) at least 20-times per 24 hours which secures ETT/TT patency and provides aggressive microbial biofilm suppression.

The proposed system and methods for combination suction-brush cleaning of ETT/TT is safer than available technology as the patient (1) does not have to be disconnected from the ventilator, which can be associated with adverse events and some patients cannot tolerate this; (2) does not require a specific cleaning procedure separate from already regularly performed in-line suctioning; (3) there are no risks for de-oxygenation periods (as patients with severe acute respiratory failure are unable to tolerate any disconnection from the breathing circuit); and (4) there are no risks for lung contamination from the brush as the brush is—together with the in-line suction system—kept always in a sterile plastic sheath. Furthermore, the disclosed system and methods do not require deflating the ventilator pressure circuit; pressure circuit deflations have been associated with partial lung collapse (atelectasis) and worsening lung performance and outcome.

### BRIEF DESCRIPTION OF THE DRAWINGS

The accompanying drawings, which are incorporated in and constitute a part of this specification, exemplify the embodiments of the present disclosure and, together with the description, serve to explain and illustrate principles of the disclosure. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1 illustrates an example of a endotracheal suctioning tube (so called in-line suctioning) connected to the patient's breathing circuit that is constructed in accordance with the principles of the present disclosure;

FIG. 2 illustrates an example of a perspective view of a suction brush segment of a cleaning catheter that is constructed in accordance with the principles of the present disclosure;

FIG. 3 illustrates an example of a cross sectional view of a suction brush segment inside of an endotracheal tube.

FIG. 4 illustrates an example of a perspective view of a suction brush segment and a diagram illustrating the brush length profile along the suction brush segment.

FIG. 5 illustrates an example of a method of cleaning a patient's endotracheal tube while the endotracheal tube is inserted inside the patient's throat that is in accordance with the principles of the present disclosure.

In the drawings, the same reference numbers and any acronyms identify elements or acts with the same or similar structure or functionality for ease of understanding and convenience. To easily identify the discussion of any particular element or act, the most significant digit or digits in a reference number refer to the Figure number in which that element is first introduced.

### DETAILED DESCRIPTION

Unless defined otherwise, technical and scientific terms used herein have the same meaning as commonly understood by one of ordinary skill in the art to which this disclosure belongs. Szycher's Dictionary of Medical Devices CRC Press, 1995, may provide useful guidance to many of the terms and phrases used herein. One skilled in the art will recognize many methods and materials similar or equivalent to those described herein, which could be used in the practice of the present disclosure. Indeed, the present disclosure is in no way limited to the methods and materials specifically described.

In some embodiments, properties such as dimensions, shapes, relative positions, and so forth, used to describe and claim certain embodiments of the disclosure are to be understood as being modified by the term “about.”

Various examples of the disclosure will now be described. The following description provides specific details for a thorough understanding and enabling description of these examples. One skilled in the relevant art will understand, however, that the disclosure may be practiced without many of these details. Likewise, one skilled in the relevant art will also understand that the disclosure can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the disclosure. Indeed, certain terms may even be emphasized below; however, any terminology intended to be interpreted in any restricted manner will be overtly and specifically defined as such in this Detailed Description section.

While this specification contains many specific implementation details, these should not be construed as limitations on the scope of any disclosures or of what may be claimed, but rather as descriptions of features specific to particular implementations of particular disclosures. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

### Overview

Disclosed are systems and methods for safely and effectively cleaning the inside of an endotracheal and tracheal tube using a combined suction and brush system. For instance, disclosed is an endotracheal suction catheter that includes a brush-suction segment that allows the catheter to mechanically brush off secretions on the inner surface of an endotracheal tube (“ETT/TT”) while simultaneously suctioning the debris. The brush-suction segment can be of various lengths (e.g., 1 inch, 0.5 inches, 2 inches, ¾ inches and other lengths) to fit within the in-line suction device's sterile compartment. The brush-suction segment can be placed at the distal tip to allow the brush segment to move through the entire ETT/TT length while suctioning.

Brush hairs emanate outwardly from the outer surface of the suction tube and may include an ovular (e.g. two opposing conical shaped funnels) profile to permit smooth ETT/TT entry and exit. The hairs may be aligned in four spiral arrays. In some embodiments, each array traverses a one-half circular turn of the suction catheter. The spiral orientation of the arrangement of the brushes along the catheter provide later (circumferential) cleaning traction to the brush action in addition to the back-and-forth cleaning motions, which occur during the insertion and retrieval motion during suctioning.

Suction channels may be spaced in-between and adjacent to the spiral brush arrays. These perforations allow aspiration of debris/secretions into the suction tube. To optimize debris/secretion aspiration and to minimize overall suction pressure loss within the in-line suction device, the channels may be grooved in V-form (with the larger channel width at the outer and smaller width at the inner wall of the suction tube). This allows the creation of a pressure gradient with lower suction pressure towards the brush surface while higher suction pressure is generated at the inner suction tube lumen.

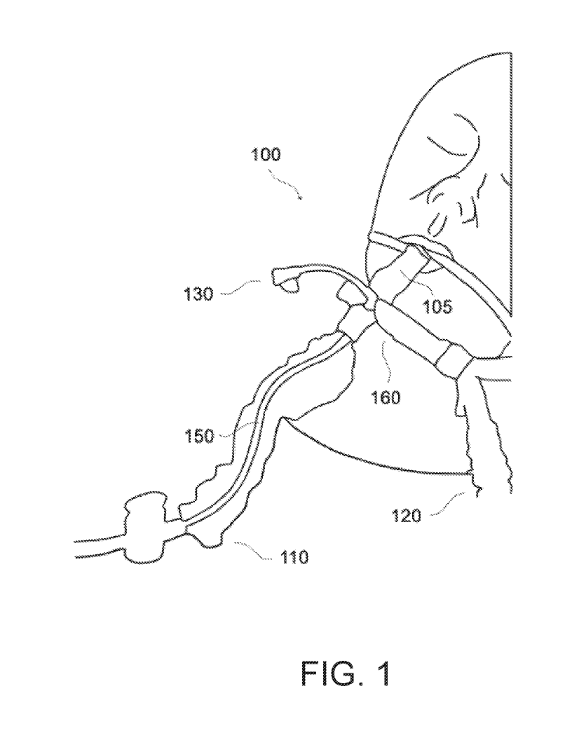

FIG. 1 illustrates an overview of a system 100 implementing the cleaning catheter 150 disclosed herein. For instance, in some embodiments, the system may include an endotracheal tube 105 inserted into the patient's throat to assist in breathing. The endotracheal tube 105 may be connected to ventilator circuit 120 and may also be connected to the cleaning catheter 150 (covered by a sterile plastic sheath 110) that can be on-demand inserted inside the endotracheal suction tube 105. The cleaning catheter 150 may be inserted by manually (or automatically) advancing the cleaning catheter 150 forward into the endotracheal tube 105. The system 100 may further include a cuff inflation tube with pilot balloon 130 which may be configured to be removably attached to the endotracheal tube 105. The system 100 may also include a collector (not shown) that is removably attached to the cleaning catheter 150.

Accordingly, the cleaning tube 150 may then be retracted and advanced while suction is applied to suction out secretions and brush off the residue that has accumulated on the inner circumference of the endotracheal tube 105. The sheath 110 ensures the catheter 150 remains in a sterile environment or free from outside contaminants when not inserted or outside the endotracheal tube 105. The back and forth action will allow the cleaning catheter 150 to appropriately brush off the secretions while extended into the endotracheal tube 105. The cleaning catheter 150 may include various markings or slidable indicators to indicate how far the cleaning catheter 150 should be inserted and removed in a brushing forward-backward fashion, so that the optimal amount of secretions and build up are removed.

In other embodiments, the ventilator circuit 120 may be disconnected or detached, and the cleaning catheter 150 may be inserted as a separate component. Accordingly, the cleaning catheter 150 may be attached to its own suction source that is controllable by the operator. The cleaning catheter 150 may attached to the endotracheal tube 105 or a T-junction tube 160 in various methods that are suitable. In some embodiments, the cleaning catheter 150 may include a connector for connecting to the T-junction tube 160.

### Suction Brush Segment

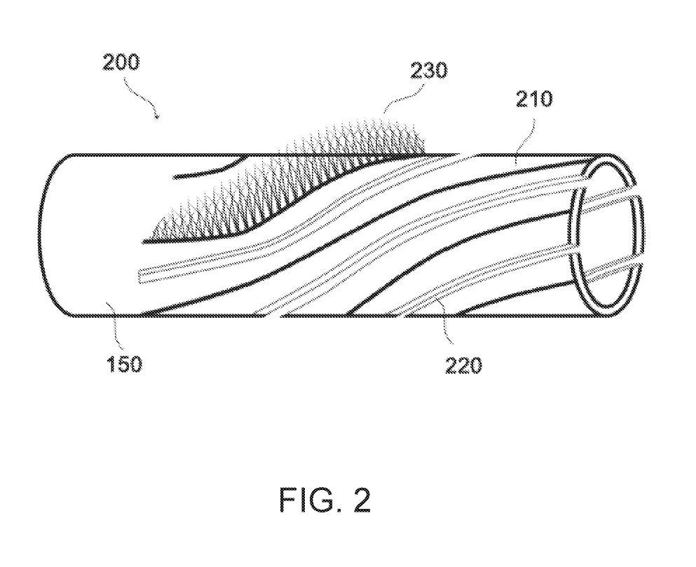

FIG. 2 illustrates a suction brush segment 200 that may be included in the midsection of the cleaning catheter 150. The suction brush segment 200 may include brush arrays 230 and suction channels 220. The brush arrays 230 may then brush off the secretions and biofilm adhering to the inside of the endotracheal tube 105 while the suction channels 220 vacuum the debris and also suction any non-adhered secretions.

The relative orientations of the brush arrays 230 and suction channels 220 may take various forms. For instance, as illustrated the brush arrays 230 may alternate with the suction channels 220. This arrangement may be advantageous because here the suction channels 220 will surround the brush arrays 230 on both sides allow any debris dislodged by the brush arrays 230 to be immediately suctioning into one of the adjacent suction channels 220. Similarly, as illustrated, the arrays 230 and channels 220 may be spiral wrapped around the cleaning catheter 150 so that motion directed along the longitudinal axis (moving the cleaning catheter 150 in and out of the endotracheal tube 105) will brush the entire inside while rotating the catheter 150 will also brush the entire inside of the endotracheal tube 105. This configuration also has the advantage of maintaining the maximum structural integrity of the cleaning catheter 150 because the channels 220 will not create a weak axis.

In other embodiments, the brush arrays 230 may be oriented to be circles around the circumference of a cross section of the cleaning catheter 150. Then suction channels 220 could also be alternated on either side of the brush arrays 230 in a similar pattern (of course, with bridges between). In other embodiments a series of brushes 230 may be positioned together proximally to suction channels 220 so that any debris that is removed and falls further distally may be suctioned by suction channels 220. The debris that is removed may be deposited into a collector that is removably (or permanently) attached to the cleaning catheter 150.

In some embodiments, the channels 220 may instead be a series of perforations that are dispersed throughout the suction brush segment 200, and the brush arrays 230 may also be dispersed between the perforations 220. In this embodiment, there may be a more uniform suctioning and brushing.

In some embodiments, the brush suction segment 220 may be positioned so that the brush arrays 230 and channels 220 extend to the distal end of the tip of the cleaning catheter 150 or near the distal tip. In these embodiments, when the cleaning catheter 150 is inserted into the endotracheal tube 105, the brush suction segment 200 will extend and reach down to the distal end of the endotracheal tube 105. This will ensure maximum cleaning. In some embodiments, when fully inserted, the cleaning catheter's 150 distal tip may extend beyond the distal tip of the endotracheal tube 150. Accordingly, in this embodiment, the suction brush segment 200 could reach beyond the distal tip of the endotracheal tube 105 as well. In some embodiments, the brush arrays 320 may reach to the end of the endotracheal tube 105 while the suction channels 220 may extend further to ensure additional debris are suctioned that fall distally and do not reach the lungs.

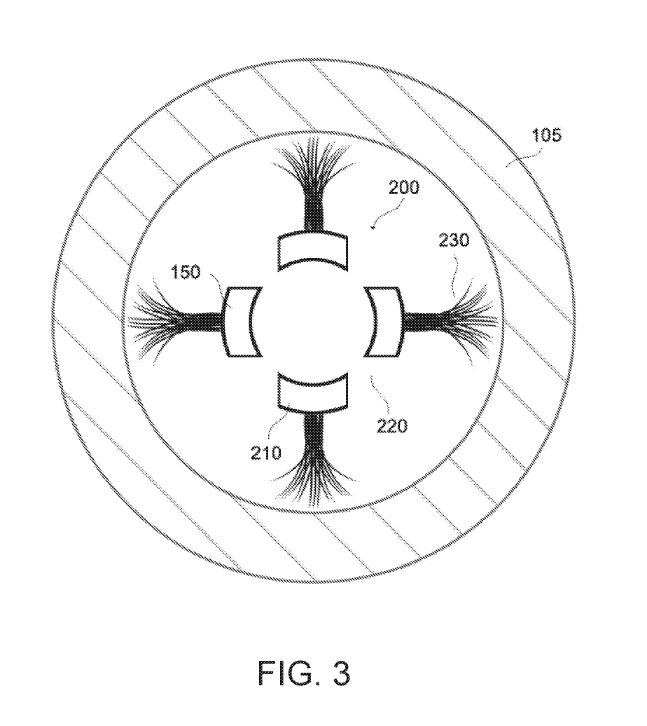

FIG. 3 illustrates a cross-sectional view of the suction brush segment 200 that is inside of an endotracheal tube 105. As illustrated, the brush hairs form the brush arrays 230 may extend to make sufficient contact with the inside of the endotracheal tube 105 to provide effective brushing and mechanical removal of debris. Additionally, the suction brush segment 200 may include the suction channels 220 that are configured to facilitate a fluid flow or communication (hereinafter referred to as “fluid communication”) with the lumen of the cleaning catheter 150.

In this embodiment, the suction channels 220 are illustrated as V-shaped suction channels. This will provide a pressure gradient along the channels that has the lowest suction pressure on the outside of the channels 220 and the greatest suction pressure towards the inside. Accordingly, the outside of the V shape will cover a greater area while the strong pressure at the inside will ensure debris that are suctioned in the channels 220 will be pulled inside the lumen of the suction channel 220 and not remain blocking it. In other embodiments various other channel geometries may be implemented including straight channels, reverse V shaped channels or other suitable configurations. In some instances, sterile water may be flushed through the in-line suction catheter to maintain the opening of the brush-suction channels and potentially flush out debris.

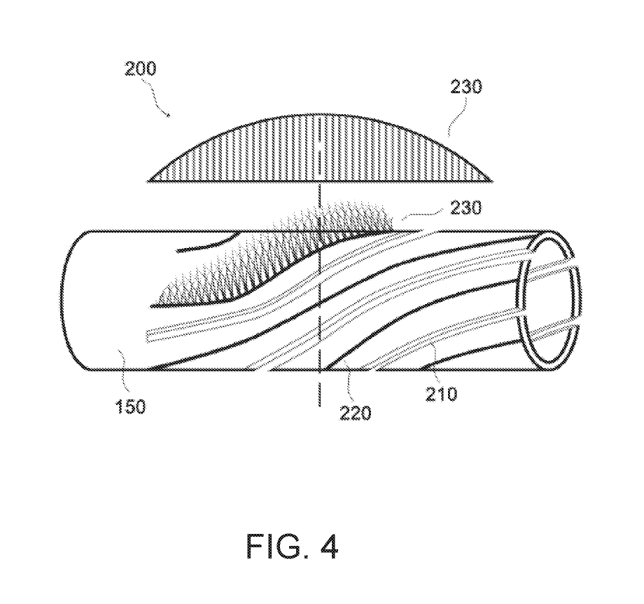

FIG. 4 illustrates an example of the suction brush segment 200 with a diagram overhead that illustrates the brush length as you move longitudinally along the brush suction segment 200. In this embodiment, the brush starts out at almost nothing, reaches maximum length in the middle, and then again tapers down to nothing at the proximal end of the brush suction segment 200. This allows little resistance to moving the brush up and down and makes for ease of inserting the cleaning catheter 150 into the endotracheal tube 105. In other embodiments, the brush 230 hairs may be of uniform length. In other embodiments, they may have an alternating length.

### Methods

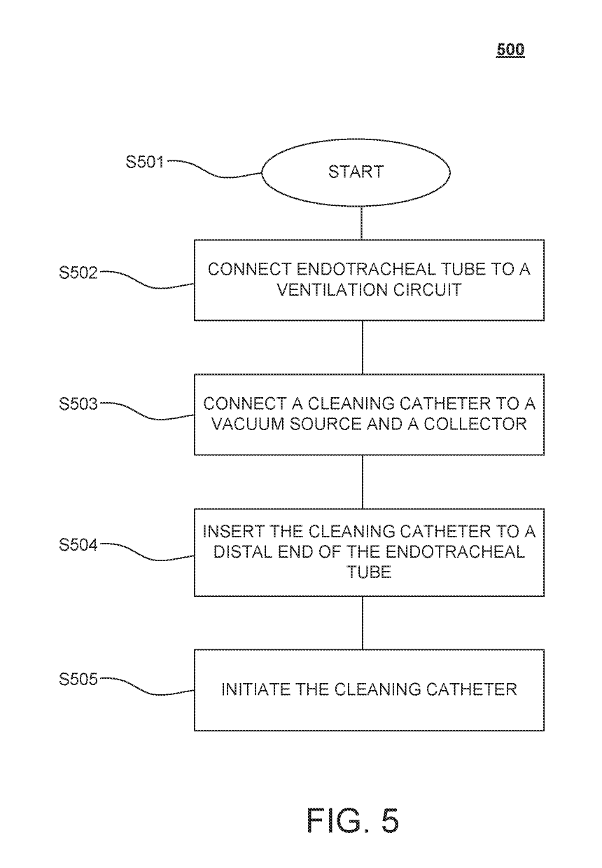

FIG. 5 illustrates a method of cleaning a patient's endotracheal tube while the endotracheal tube is inserted inside the patient's throat. The method includes connecting the endotracheal tube to a ventilation circuit to provide assisted breathing to the patient (S502); connecting a cleaning catheter with a suction brush segment to a vacuum source and a collector (S503); inserting the cleaning catheter so that the suction brush segment is positioned at or near a the beginning (proximal end) of the endotracheal tube (S504); and initiating the vacuum source and manually moving the cleaning catheter inside of the endotracheal tube to remove debris (S505).

The ventilation circuit may be configured to be temporarily disconnected during insertion of the cleaning catheter. The suction brush segment may be configured to be removably connectable to the cleaning catheter and may be replaced after use. The brush segment may be configured to be attached to a suction catheter to assemble the cleaning catheter prior to use.

As discussed herein, the cleaning catheter 150 may be inserted into the endotracheal tube 105 intermittently (or continuously) to suction and brush the inside of the endotracheal tube 105. The caregiver may use different combinations of motions to clean the inside of the tube 105 including spiral turns, in and out motions, or combinations.

The cleaning catheter 150 may be assembled in various methods. In some embodiments, the cleaning catheter 150 may be a brush 230 attachment to an existing suction catheter. In other embodiments, the entire suction brush segment 200 may be attached to an existing suction catheter. In still other embodiments, the cleaning catheter may be manufactured separately, and then assembled with a sheath, connector and other components in order to be connectable to a T-junction tube 160 for a respiration system.

### SELECTED EMBODIMENTS

### Embodiment 1

A cleaning system for cleaning the inside of an endotracheal tube, the cleaning system comprising: a connection port for connecting to a vacuum source and a collector; a cleaning catheter that is configured to be inserted inside of a endotracheal or tracheal tube; and a brush suction segment on a mid-section of the cleaning catheters, wherein the brush suction segment comprises suction channels and brush arrays, wherein the suction channels are configured to be in a fluid communication with a lumen of the endotracheal tube, wherein the lumen of the endotracheal tube is configured to be in fluid communication with the connection port, and wherein the connection port and the cleaning catheter are configured to be removably connected to each other.

### Embodiment 2

The cleaning system of embodiment 1, wherein the suction channels comprise a V shape with a larger opening of the suction channels facing an outside of the cleaning catheter.

### Embodiment 3

The cleaning system of embodiment 1, wherein the brush arrays and suction channels are configured to be formed as alternating spiral arrays that extend along the brush suction segment.

### Embodiment 4

The cleaning system of embodiment 1, wherein the suction channels comprise a series of holes.

### Embodiment 5

The cleaning system of embodiment 1, wherein the brush arrays comprise a series of patches dispersed around a circumference of the brush suction segment.

### Embodiment 6

The cleaning system of embodiment 1, wherein the brush arrays and suction channels form alternating circles around the circumference of the brush suction segment.

### Embodiment 7

The cleaning system of embodiment 3, wherein the brush arrays and suction channels travel a half turn or a full turn around the brush suction segment from a distal end to a proximal end of the endotracheal tube.

### Embodiment 8

The cleaning system of embodiment 1, wherein the vacuum source is configured to apply suction to the lumen via the suction channels.

### Embodiment 9

The cleaning system of embodiment 8, wherein debris that is collected from the endotracheal tube by the suction channels is deposited into the collector.

### Embodiment 10

A method of cleaning a patient's endotracheal tube while the endotracheal tube is inserted inside the patient's throat, the method comprising: connecting the endotracheal tube to a ventilation circuit to provide assisted breathing to the patient; connecting a cleaning catheter with a suction brush segment to a vacuum source and a collector; inserting the cleaning catheter so that the suction brush segment is positioned at or near a proximal end of the endotracheal tube; and initiating the vacuum source and manually moving the cleaning catheter inside of the endotracheal tube to remove debris.

### Embodiment 11

The method of embodiment 10, wherein the ventilation circuit is configured to be temporarily disconnected during insertion of the cleaning catheter.

### Embodiment 12

The method of embodiment 10, wherein the suction brush segment is configured to be removably connectable to the cleaning catheter and may be replaced after use.

### Embodiment 13

The method of embodiment 10, wherein the brush segment is configured to be attached to a suction catheter to assemble the cleaning catheter prior to use.

### Embodiment 14

A cleaning catheter for cleaning the inside of an endotracheal tube comprising: a connection port for connecting to a vacuum source and a collector; a tube portion that is sized to be inserted inside of a endotracheal tube; a brush suction segment on a distal end of the tube portion, the brush suction segment including suction channels and brush arrays, the suction channels in fluid communication with a lumen, wherein the lumen is in fluid communication with the connection port.

### Embodiment 15

The cleaning catheter of embodiment 14, wherein the suction channels are V shaped with a larger opening facing an outside of the cleaning catheter.

### Embodiment 16

The cleaning catheter of embodiment 14, wherein the brush arrays and the suction channels are formed as alternating spiral arrays that extend along the brush suction segment.

### Embodiment 17

The cleaning catheter of embodiment 14, wherein the suction channels are a series of holes.

### Embodiment 18

The cleaning catheter of embodiment 14, wherein the brush arrays are a series of patches dispersed around the circumference of the brush suction segment.

### Embodiment 19

The cleaning catheter of embodiment 14, wherein the brush arrays and suction channels form alternating circles around the circumference of the brush suction segment.

### Embodiment 20

The cleaning catheter of embodiment 16, wherein the brush arrays and suction channels are formed as a half turn or a full turn around the brush suction segment from a distal end to a proximal end of the endotracheal tube.

### CONCLUSION

The various methods and techniques described above provide a number of ways to carry out the disclosure. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the disclosure has been disclosed in the context of certain embodiments and examples, it will be understood by those skilled in the art that the embodiments of the disclosure extend beyond the specifically disclosed embodiments to other alternative embodiments and/or uses and modifications and equivalents thereof.

In some embodiments, the terms “a” and “an” and “the” and similar references used in the context of describing a particular embodiment of the disclosure (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the disclosure and does not pose a limitation on the scope of the disclosure otherwise claimed. No language in the specification should be construed as indicating any non-claimed element essential to the practice of the disclosure.

Certain embodiments of this disclosure are described herein. Variations on those embodiments will become apparent to those of ordinary skill in the art upon reading the foregoing description. It is contemplated that skilled artisans can employ such variations as appropriate, and the disclosure can be practiced otherwise than specifically described herein. Accordingly, many embodiments of this disclosure include all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the disclosure disclosed herein are illustrative of the principles of the embodiments of the disclosure. Other modifications that can be employed can be within the scope of the disclosure. Thus, by way of example, but not of limitation, alternative configurations of the embodiments of the disclosure can be utilized in accordance with the teachings herein. Accordingly, embodiments of the present disclosure are not limited to that precisely as shown and described.

What is claimed is:

1. A cleaning system for cleaning the inside of an endotracheal tube, the cleaning system comprising:
a connection port for connecting to a vacuum source and a collector;
a cleaning catheter that is configured to be inserted inside of an endotracheal or tracheal tube; and
a brush suction segment on a mid-section of the cleaning catheters,
wherein the brush suction segment comprises suction channels and brush arrays,
wherein the suction channels are configured to be in a fluid communication with a lumen of the endotracheal tube,
wherein the lumen of the endotracheal tube is configured to be in fluid communication with the connection port, and
wherein the connection port and the cleaning catheter are configured to be removably connected to each other.

2. The cleaning system of claim 1, wherein the suction channels comprise a V shape with a larger opening of the suction channels facing an outside of the cleaning catheter.

3. The cleaning system of claim 1, wherein the brush arrays and suction channels are configured to be formed as alternating spiral arrays that extend along the brush suction segment.

4. The cleaning system of claim 1, wherein the suction channels comprise a series of holes.

5. The cleaning system of claim 1, wherein the brush arrays comprise a series of patches dispersed around a circumference of the brush suction segment.

6. The cleaning system of claim 1, wherein the brush arrays and suction channels form alternating circles around the circumference of the brush suction segment.

7. The cleaning system of claim 3, wherein the brush arrays and suction channels travel a half turn or a full turn around the brush suction segment from a distal end to a proximal end of the endotracheal tube.

8. The cleaning system of claim 1, wherein the vacuum source is configured to apply suction to the lumen via the suction channels.

9. The cleaning system of claim 8, wherein debris that is collected from the endotracheal tube by the suction channels is deposited into the collector.

10. A method of cleaning a patient's endotracheal tube while the endotracheal tube is inserted inside the patient's throat, the method comprising:
connecting the endotracheal tube to a ventilation circuit to provide assisted breathing to the patient;
connecting a cleaning catheter with a suction brush segment to a vacuum source and a collector;
inserting the cleaning catheter so that the suction brush segment is positioned at or near a proximal end of the endotracheal tube; and
initiating the vacuum source and manually moving the cleaning catheter inside of the endotracheal tube to remove debris.

11. The method of claim 10, wherein the ventilation circuit is configured to be temporarily disconnected during insertion of the cleaning catheter.

12. The method of claim 10, wherein the suction brush segment is configured to be removably connectable to the cleaning catheter and may be replaced after use.

13. The method of claim 10, wherein the brush segment is configured to be attached to a suction catheter to assemble the cleaning catheter prior to use.

14. A cleaning catheter for cleaning the inside of an endotracheal tube comprising:
a connection port for connecting to a vacuum source and a collector;
a tube portion that is sized to be inserted inside of a endotracheal tube;
a brush suction segment on a distal end of the tube portion, the brush suction segment including suction channels and brush arrays, the suction channels in fluid communication with a lumen, wherein the lumen is in fluid communication with the connection port.

15. The cleaning catheter of claim 14, wherein the suction channels are V shaped with a larger opening facing an outside of the cleaning catheter.

16. The cleaning catheter of claim 14, wherein the brush arrays and the suction channels are formed as alternating spiral arrays that extend along the brush suction segment.

17. The cleaning catheter of claim 14, wherein the suction channels are a series of holes.

18. The cleaning catheter of claim 14, wherein the brush arrays are a series of patches dispersed around the circumference of the brush suction segment.

19. The cleaning catheter of claim 14, wherein the brush arrays and suction channels form alternating circles around the circumference of the brush suction segment.

20. The cleaning catheter of claim 16, wherein the brush arrays and suction channels are formed as a half turn or a full turn around the brush suction segment from a distal end to a proximal end of the endotracheal tube.
